(19) United States
(12) Patent Application Publication
(10) Pub. No.: US 2006/0275838 A1
(43) Pub. Date: Dec. 7, 2006

(54) Compositions and methods for inhibiting liver stellate cell growth

(76) Inventors: Ranjit Ray, Saint Louis, MO (US); Ratna Ray, Saint Louis, MO (US); Arnab Basu, Natick, MA (US); Yie-Hwa Chang, Saint Louis, MO (US)

Correspondence Address: SAINT LOUIS UNIVERSITY;OFFICE OF INNOVATION AND INTELLECTUAL PROPERTY, 3556 CAROLINE MALL, SUITE C208, ST. LOUIS, MO, 63104, US

(21) Appl. No.: 11/436,384

(22) Filed: May 18, 2006

Related U.S. Application Data
(63) Continuation-in-part of application No. 10/888,962, filed on Jul. 9, 2004.
(60) Provisional application No. 60/487,126, filed on Jul. 12, 2003.

(51) Int. Cl. G01N 33/567; C07H 21/04; C12P 21/06; C07K 14/475; C12N 9/99

(52) U.S. Cl. 435/7.2; 435/69.1; 435/320.1; 435/325; 530/350; 536/23.5

(57) ABSTRACT

The present invention provides compositions and methods for selectively inhibiting the proliferation of stellate cells, which are important for the development of liver fibrosis upon liver injury. The invention describes conditioned media from immortalized hepatocytes as containing a death factor that induces apoptosis of activated liver stellate cells. This pro-apoptotic activity is shown to be associated with the peptide sequence of the actin depolymerizing molecule gelsolin and or fragments thereof. The apoptotic activity is increased upon incubation of immunoglobulins with the stellate death factor.

### PARENT CASE TEXT

This application claims benefit of priority to U.S. Provisional Patent Application No. 60/487,126, filed Jul. 12, 2003, and also benefit of priority to a continuation-in-part of U.S. application Ser. No. 10/888,962 filed on Jul. 9, 2004.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

1. Field of the Invention

This invention relates generally to compositions and methods of treating liver fibrosis or cirrhosis. Specifically, the invention is directed to compositions and methods for killing liver stellate cells.

2. Description of the Related Art

According to the American Liver Foundation, over 300,000 Americans are hospitalized each year for cirrhosis of the liver. The primary causes of cirrhosis are alcohol abuse and chronic hepatitis C. To date, approximately 3.9 million Americans suffer from Hepatitis C. It is also estimated that 18,000 people are in need of liver transplants, which are in woefully short supply. Thus, it is essential to saving lives that new medical treatments for preventing and reversing liver cirrhosis are developed.

Hepatitis C virus (HCV) is a major causative agent of acute and chronic hepatitis, which may lead to liver cirrhosis and hepatocellular carcinoma (Choo, Q. L. et al, 1989; Di Bisceglie, A. M. 1997; Saito I. et al 1990). Natural immune responses are not capable of terminating HCV infection in most patients. Furthermore, neither a vaccine nor any other means of very effective therapy is available to control HCV (McHutchison et al., 1998). Immune evasion and a quasispecies nature are prominent features of HCV (Farci et al., 1992; Weiner et al., 1992; Purcell, 1994). The molecular mechanisms whereby HCV circumvents the immune response, persists, and causes chronic liver disease is not well understood. However, these processes would likely require immune mediated factors, and the interaction of viral proteins with cellular factors (Rehermann and Chisari, 2000).

HCV contains a single positive-stranded RNA as its genome. HCV genome encodes a precursor polypeptide of −3,000 amino acids. This precursor polypeptide is cleaved by both host and viral proteases to at least 10 individual proteins: C, E1, E2, p7, NS2, NS3, NS4A, NS4B, NS5A, and NS5B (Clarke, B. 1997). Diverse functional activities of the HCV core protein have already been noted by a number of investigators (Ray and Ray, 2001—FEMS). Our own work and the results from other laboratories suggest that the core protein has multifunctional activities. These include encapsidation of viral RNA, a regulatory effect on cellular and unrelated viral promoters, interactions with a number of cellular proteins, a modulatory role in programmed cell death or apoptosis under certain conditions, involvement in cell growth promotion and immortalization, induction of HCC in transgenic mice, and a possible immunoregulatory role. These intriguing properties suggest that the core protein, in concert with diverse cellular factors, may contribute to pathogenesis during persistent HCV infection.

Hepatic stellate cells (HSC) constitute approximately 15% of the total number of resident liver cells, and are the pivotal cell type involved in the development of hepatic fibrosis (McGee J O, J Pathol; 106, 1972; McGee J O, Lab Invest; 26:429-440, 1972). Following liver injury of any etiology, HSC are activated from quiescent cells into proliferative, fibrogenic, and contractile myofibroblasts (Friedman, 2000, and Proc Natl Acad Sci USA 1985;82: 8681-8685, and Rockey D C, Submicrosc Cytol Pathol 1992;24:193-203.). The survival of activated HSC in liver injury is dependent on soluble growth factors and cytokines, and on components of the fibrotic matrix (Iredale, 2001).

Liver fibrosis is a central feature of the majority of chronic liver injuries due to metabolic, genetic, viral, and cholestatic diseases. It results in distortion of the liver architecture (cirrhosis), which is associated with disturbance of liver function and significant morbidity and mortality (Friedman SL. N Engl J Med., 328:1828-1835, 1993). During the liver injury these cells are activated and the process involves cell proliferation and acquisition of fibrogenic and contractile capacity. Liver hepatocytes play an important role in this activation (Smith et al; 2003; Hepatology). The resolution of hepatic fibrosis is associated with the remodeling of the excess liver matrix and may result in restitution of near normal liver architecture in patients (J. F. Dufour, et al Dig Dis Sci. 1998, 43 2573-2576; J. F. Dufour, et al. Ann Intern Me, 199,7 127, 981-98; Kaplan, R. A. et al. Ann Intern Med. 1997, 126, 682-688) and experimental animal models (G. Abdel-Aziz, 1990). An essential element of this recovery process is the apoptosis of activated HSC (J. P. Iredale et al J Clin Invest. 1998, 102 538-549). Understanding the mechanisms of HSC apoptosis might provide insight into novel therapeutic approaches to treat advanced hepatic fibrosis. HSC apoptosis are shown to be induced by activated Kupffer cells through a novel mechanism (Fischer R, et al. Gastroenterology. 2002;123:845-61) and by ligands of the peripheral type benzodiazepine receptor (Fischer R, et al. Gastroenterology. 2001;120:1212-1226). However, very little is known about the role of hepatocytes for HSC apoptosis. Murine hepatocytes have been shown to secrete an inducing protein that selectively causes apoptosis in liver (Ikeda et al, Immunology, 2003, 108,116-122). Hepatic stellate cells, when isolated and grown on plastic surface, spontaneously undergo activation. These culture induced activated stellate cells have been extensively studied as a model cell line of liver fibrogenesis.

The inventors have sought to address the issue of liver homeostasis and disease, and in particular mediators of stellate cells which may be secreted by hepatocytes. Understanding these mediators and their pathways will offer new avenues for therapeutic strategies to combat liver disease particularly those involving stellate cells such as cirrhosis.

### SUMMARY OF THE INVENTION

The inventors have made the surprising discovery that conditioned medium from immortalized hepatocytes (“immortalized hepatocyte-conditioned medium”) contains a death factor, which comprises a biochemical activity, which is the promotion of apoptosis of a liver stellate cell (“pro-apoptotic activity”). The inventors have further demonstrated through peptide mass fingerprinting of the purified soluble mediator from conditioned media (“CM”), that the actin depolymerizing protein gelsolin in a fragmented form, plays a role in apoptosis of LX2 cells. Furthermore the cytotoxic effect can be enhanced by the binding of immunoglobulins to these gelsolin fragments.

Purification of the soluble mediator by ion-exchange chromatography, and analysis by mass spectrometric (LC/MS) suggested that the actin binding molecule gelsolin is present as an intact protein and also as polypeptide fragments, including fragments of 23, 25, 46 and 50 kDa (kDa=10³ Daltons). Gelsolin is highly conserved in vertebrates and exists in two isoforms, a cytoplasmic and an extracellular variant, generated by alternative splicing.

The inventors demonstrated that the pro-apoptotic activity is not affected by treatment with (a) metallo-protease inhibitors or (b) antibodies to known pro-apoptotic factors, such as TRAIL and Fas ligand. Further studies indicate that stellate cell death occurs through apoptosis. Conditioned media from immortalized hepatocytes (IH) increased the expression of TRAIL receptors on LX2 cell surface, and induced apoptosis by a caspase dependent mechanism.

The inventors also made the surprising discovered that the binding of immunoglobulins to fragmented gelsolin, greatly enhances the pro-apoptotic activity. The addition of an IgGla isotype of a mouse monoclonal antibody, directed to an epitope on the carboxy terminal region of gelsolin, significantly enhanced CM mediated HSC toxicity. Further analysis indicated that the mouse monoclonal antibody recognizes fragmented gelsolin of different molecular sizes (28-93 kDa) present in the CM. It was also determined that sera from 4 of 12 human patients chronically infected with hepatitis C contained antibodies to fragmented gelsolin. These results suggest an important role for an immune meditated response to fragmented gelsolin in chronic liver injury.

Therefore, an object of this invention is a stellate death factor capable of inhibiting the proliferation of stellate cells, by inducing apoptosis in stellate cells, comprised of the actin depolymerizing molecule gelsolin, and or, fragments thereof, including those identified by the inventors of approximately 23, 25, 46, and 50 kDa.

In another embodiment, the object of this invention is a method of inhibiting the proliferation of stellate cells, by inducing apoptosis in stellate cells, by contacting the stellate cells with a stellate death factor. The death factor, comprising gelsolin, and or, one or more fragments of gelsolin, may be administered as a composition, such as immortalized hepatocyte conditioned media, or media conditioned by other immortal hepatocytes or hepatoma cells. Alternatively, the death factor may be purified, such as according to an ion exchange process or according to other biochemical isolation methods from conditioned media, to be administered to stellate cells to induce apoptosis.

In another embodiment, the object of this invention is a method of inhibiting the proliferation of stellate cells, by inducing apoptosis in stellate cells, by contacting the stellate cells with a composition comprising the stellate death factor gelsolin and or fragments of gelsolin, and an immunoglobulin directed against gelsolin. Alternatively, gelsolin or fragments of gelsolin may be modified so as to elicit an immune response from the host patient.

In another embodiment, the invention is drawn to a method of manufacturing a stellate cell death factor, comprising the steps of (a) conditioning media with an immortalized hepatocyte or hepatoma cell to produce conditioned media (“CM”) which comprises the stellate cell death factor, and then optionally (b) applying the CM to an anion exchange column, (c) applying the resultant flow-through to a first cation exchange column, (d) eluting a fraction comprising the stellate cell death factor with 0.5 M NaCl, (e) dialyzing eluant into a first buffer, (f) applying dialysate onto a second cation exchange column, (g) and eluting fractions comprising the stellate cell death factor using a 50 to 500 nM NaCl gradient. Similarly, stellate cell death factor may be manufactured from immortalized hepatocyte or hepatoma cell lysates.

In yet another embodiment, the invention is drawn to a method of manufacturing a stellate cell death factor, comprising a) purifying gelsolin by any number of biochemical methods, and b) subjecting gelsolin to enzymatic proteolysis to produce one or more polypeptide fragments including those identified by the inventors of 23, 25, 46, and 50 kDa.

In yet another embodiment, the invention is drawn to a method of manufacturing a stellate cell death factor, comprising recombinant DNA technology to produce a polypeptide of amino acid sequence with homology to gelsolin and pro-apoptotic activity against stellate liver cells.

It is envisioned that the instant stellate cell death factor (supra) may be administered to stellate cells in vivo, in a pharmaceutically acceptable formulation, as a therapy for the treatment of a hepatic fibrosis disease or liver cirrhosis.

### BRIEF DESCRIPTION OF THE DRAWINGS

FIG. 1: Growth of human stellate cells in conditioned medium from immortalized hepatocytes (TPH −1, passage 10 or passage 50) and medium from THLE as a negative control. The growth was measured using a Cell Titer TM AQueous Non-Radioactive Cell Proliferation Assay (Promega).

FIG. 2: Growth of rat stellate cells in conditioned medium from immortalized hepatocytes (TPH −1, passage 50) and medium from SABM as a negative control. The growth was measured using a Cell Titer TM AQueous Non-Radioactive Cell Proliferation Assay (Promega).

FIG. 3: Apoptosis of activated human liver stellate cells (“LX2 cells”) by conditioned medium. Panel A: Analysis of DNA fragmentation in” LX2 cells incubated with conditioned medium from THLE cells as a negative control (lane 1), THLE-core (lane 2), and from TPH (lane 3). Panel B: Quantification of DNA fragmentation in LX2 cells upon incubation with conditioned medium from TPH −1, passage 50 or THLE (negative control). DNA fragmentation was quantified from cytosolic oligonucleotides-bound DNA using ELISA (Roche).

FIG. 4: Panel A: Identification of HCV core transfected primary human hepatocytes and stellate cells in culture. Hepatocytes were identified by indirect immunofluorescence with a specific MAb Hep Par. Activated stellate cells were identified using a MAb to α-smooth muscle actin. Effect of soluble mediator in conditioned medium of TPH on stellate cell growth. Panel B: LX2 cells were treated with CM from early (▪-▪) and late passage (▴-▴) TPH. LX2 cells were similarly treated with SABM (●-●) for comparison. Rat stellate cells were similarly treated with CM from TPH cells (▪-▪). Rat stellate cells were similarly treated with SABM (●-●) for comparison. Cell viability was assessed from triplicate culture wells by Cell Titer 96 Aqueous non-radioactive cell proliferation kit (Promega) at different time points and presented as mean values. Panel C: Conditioned medium from TPH exhibited a dose dependent effect on LX2 cell viability.

FIG. 5: Panel A: Soluble mediator from TPH induces apoptosis in LX2 cells. Analysis for DNA fragmentation of LX2 cells following treatment with SABM (lane 1), CM from late passage TPH (lane 2) or after culture of LX2 and TPH in dual chamber Transwell dish (lane 3). DNA extracted from cells was analyzed by 1.6% agarose gel electrophoresis. Panel B: Quantitation of DNA fragmentation in LX2 cells. CM from TPH or SABM treated LX2 cells were analyzed for cytosolic oligonucleosome-bound DNA by ELISA (Roche).

FIG. 6: Induction of TRAIL receptors by CM. FACS analysis was performed to determine the expression levels of TRAIL-R1 and TRAIL-R2 on LX2 cell surface with and without CM treatment. Cells were treated with specific monoclonal antibody conjugated to phycoerythrin for FACS analysis. The mean fluorescence intensities of negative control (grey area), isotype control (solid line), and antibody treated (dotted line) cells are shown.

FIG. 7: Panel A: Expression level of DR4, and DR5 in TPH CM treated LX2 cells and in control cells. The level of cellular actin was used as an internal control. Arrows on the right indicate respective proteins. Molecular weights of the respective proteins were verified from the position of prestained molecular weight markers (Invitrogen). Panel B: Reciprocal antibody dilutions of TRAIL receptors. Apoptotic cell death was analysed by ELISA from quantitation of cytosolic oligonucleosome-bound DNA in control and CM treated LX2 cells, prior incubated with different doses anti-TRAIL-R1 (DR4) and/or anti-TRAIL-R2 (DR5) antibody. Each antibody represented 50% concentration when both were used in combination.

FIG. 8: Involvement of caspase dependent apoptotic signaling pathway in CM treated LX2 cells. Western blot analysis for expression status of caspase 8 precursor (panel A), caspase-7 (panel B), caspase-3 (panel C), and PARP (panel D) in control and CM treated LX2 cells. Cellular actin was used as an internal control to verify the level of protein load in each lane. Arrows on the right indicate respective proteins. The molecular weights of the specific protein bands were verified from the positions of pre-stained molecular weight markers (Invitrogen).

FIG. 9: Depiction of a representative protocol for purifying the pro-apoptotic activity from conditioned medium.

FIG. 10: Analyses of partially purified stellate death factor. Conditioned media of immortalized hepatocytes was subjected to ion exchange chromatography and the pro-apoptosis inducing fraction analyzed by SDS-PAGE as shown here. Individual protein bands A2-A8 were cut from the gel and analyzed by peptide mass fingerprinting (LC-MS). Amino acid sequence homology with NCBI database indicated: A2 and A3 contain human albumin, A4 contains ezrin and bands A5-A8 contain 50, 46, 25, and 23 kDa fragments of gelsolin respectively.

FIG. 11: Gelsolin fragments induce apoptosis in LX2 cells. Panel A. Cell death ELISA following treatment of quiescent and activated LX2 cells. Cells were treated for 24 h by CM with or without incubation with HCV infected human serum or gelsolin specific monoclonal antibody (GS-2C4). Panel B. FACS analysis for Fc receptor expression in LX2 cells. The mean fluorescence intensities of negative control (grey area), isotype control (white line), and antibody treated (dotted line) cells are shown. Panel C. Immunoprecipitation of CM and cell lysates with human sera or monoclonal antibody, followed by Western blot analysis using a monoclonal antibody to gelsolin (GS-2C4). Cell lysates from extensively washed IH (right lane) were subjected to Western blot analysis only.

### DETAILED DESCRIPTION OF THE INVENTION

The inventors have shown previously that hepatitis C virus (HCV) core protein immortalizes primary human hepatocytes. A role of the immortalized hepatocytes (IH) on mammalian (e.g., human and rat) hepatic stellate cell growth regulation is herein disclosed. Preferential growth of IH was observed when cocultured with activated mammalian liver stellate cells. Further studies disclosed herein suggest that mammalian stellate cells undergo apoptosis when grown together with IH in a dual chamber or when incubated with conditioned medium from IH. However, mammalian liver stellate cell death was not observed when incubated with conditioned medium from non-hepatic neoplastic cell lines or from an epithelial cell line, indicating that IH generate soluble mediator(s) for stellate cell cytotoxicity. The effect of hepatocyte conditioned media on stellate cells was not due to FasL, TGF-β, TRAIL, IL-7 or IL-8, as neutralizing antibodies to these cytokines/growth factors did not prevent cell death. Neither was TIMP or TNF related cytokines involved, as expression of these cytokines was unchanged when examined by cytokine array. Subsequent analysis suggested that treatment of mammalian liver stellate cells with conditioned medium from IH increases TRAIL receptors (e.g., DR4 and DR5), and apoptosis was found to be associated with the activation of several caspases and the cleavage of PARP. Stellate cell death factor released by IH in conditioned medium was found to be heat labile. Furthermore, CM from IH was fractioned by chorography procedures and pro-apoptosis inducing fractions analyzed by peptide mass fingerprinting (LC-MS). Peptides, of approximately 23, 25, 46, and 50 kDa, were found to correspond to fragments of gelsolin. Fragmented gelsolin was also identified in CM by western blot analysis. Furthermore, it was surprisingly found that the addition of a monoclonal antibody directed against an epitope present on gelsolin enhanced their pro-apoptotic effect. Sera from 4 of 12 patients with chronic HCV infection were also shown to contain antibodies directed against fragmented gelsolin. Together, these observations suggest that the control of activated stellate cell growth by immortalized hepatocytes may be mediated through gelsolin, or fragments of gelsolin, produced through either alternative splicing or post-translation or extracellular modification. In addition, the pro-apoptotic effect may be enhanced by the binding of immunoglobulins to fragmented gelsolin.

Therefore, the invention is drawn to (1) a stellate cell death factor comprising a pro-apoptotic activity, which may be contained in or derived from immortalized hepatocyte conditioned media, and is, or is associated with, gelsolin or fragments of gelsolin, (2) methods of killing stellate cells by apoptosis by administering to stellate cells the stellate cell death factor, (3) methods of manufacturing a liver stellate cell death factor and (4) methods of enhancing the cytotoxic activity by binding immunoglobulins to a stellate death factor. The stellate cell may be ex vivo or in a patient who suffers from a hepatic fibrosis disease, of which cirrhosis of the liver is an example.

The term “death factor” means any agent that promotes the killing of any cell. Killing may be by necrosis or apoptosis (programmed cell death). A “stellate cell death factor” promotes the preferential killing of liver stellate cells relative to hepatocytes. A death factor may be a metal, enzyme or other polypeptide, protein, ternary complex of biological molecules, peptide fragment, nucleic acid or polynucleotide, lipid, fatty acid, carbohydrate, secondary messenger molecule, ion, atom, or compound.

The term “pro-apoptotic activity” means the act of, or the capability of, promoting or inducing apoptosis (a.k.a. programmed cell death), which is characterized by cellular blebbing and DNA laddering. Pro-apoptotic activity may reside inherently in a biological molecule, such as a polypeptide, or a ternary complex comprising a polypeptide. Pro-apoptotic activity may reside inherently with the instant gelsolin protein or a fragment thereof.

The term “gelsolin”, refers to the actin binding molecule gelsolin, also know as actin depolymerizing factor (ADF), Brevin, and AGEL and is equivalent to human precursor gelsolin. It includes polypeptides belonging to the gelsolin superfamily of proteins. Table 1 provides the GenBank accession numbers of exemplary gelsolin polypeptides, as well as a summary of the sequence identities of gelsolin related molecules between several mammals. A preferred gelsolin comprises a sequence that is at least 93% identical to the human precursor gelsolin sequence as set forth in SEQ ID NO:1.

Sequence identity or percent identity is intended to mean the percentage of same residues between two sequences. The reference sequence is human precursor gelsolin. In all of the sequence comparisons, the two sequences being compared are aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5 diagonals saved in pairwise alignment=5. The residue weight table used for the alignment program is PAM250 (Dayhoff et al., in Atlas of Protein Sequence and Structure, Dayhoff, Ed., NBRF, Washington, Vol. 5, suppl. 3, p. 345, 1978).

Table 1 shows the calculations of identity for comparisons of gelsolin from various mammalian species relative to human precursor gelsolin.

TABLE 1
Percent Identity of gelsolin sequences

| Species | Accession number | Percent Identity |
|---|---|---|
| Human precursor form | NP_000168 | 100 |
| of gelsolin | | |
| Human gelsolin b | NP_937895 | 100 |
| Pig | CAA32077 | 95 |
| Mouse | NP_666232 | 93 |
| Rat | AAH79472 | 93 |

The term “immortalized hepatocyte” means any cell that is capable of secreting albumin or gelsolin, and can survive in culture for at least 5 weeks. Non-limiting examples of immortalized hepatocytes include transfected primary human hepatocytes (“TPH cells”), which are primary hepatocytes that have been transformed with a DNA encoding all or part of a hepatitis C viral core protein, and immortal hepatocytes, hepatomas or hepatocarcinoma cells. A preferred immortalized hepatocyte expresses telomerase.

The term “immortalized hepatocyte conditioned media” means any tissue culture medium in which immortalized hepatocytes have been grown for any period of time. A preferred immortalized hepatocyte medium contains a stellate cell death factor.

The term “inhibiting proliferation” means inhibiting the growth or division of a cell, inhibiting the transit by a cell through the cell cycle, preventing a cell from exiting GO of the cell cycle, inducing a cell to become quiescent, killing a cell, promoting the death of a cell, inducing apoptosis of a cell, reducing the rate of an increase in cell number in a population of cells, or decreasing the number of cells in a population.

The term “immunoglobulin” means any immunoglobulin which is cable of enhancing the cytotoxic effect of a stellate death factor. Non-limiting examples of immunoglobulins include those capable of binding Fc receptors such as the IgG isotype in particular IgG1 and IgG3.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Immortalized Hepatocytes Induce Stellate Cell Apoptosis

We have previously shown that hepatitis C virus (HCV) core protein immortalizes primary human hepatocytes (Ray et al., 2000; Basu et al., 2002). In this study, we investigated the role of the transfected primary hepatocytes (TPH) on regulation of hepatic stellate cell growth. Preferential growth of the immortalized hepatocytes (1H) was observed when co-cultured with an activated hepatic stellate cell (LX2) line. Further studies suggested that LX2 cells undergo apoptosis when grown with TPH cells in dual chambers or incubated with conditioned medium from TPH cells. However, LX2 cell death was not observed when incubated with conditioned media from a number of non-hepatic epithelial cells (HeLa, BHK, or MCF-7), indicating that TPH cells secrete a specific death factor. The effect of the conditioned media from TPH on LX2 cells was not due to FasL, TGF-beta, TRAIL, IL-7 or IL-8, as neutralizing antibodies to these cytokine growth factors did not prevent LX2 cell death. LX2 cell death factor released by immortalized TPH was enriched and purified by employing biochemical and analytical separation procedures. The secretory death factor was found to be, or be associated with gelsolin in a fragmented form. The inhibitory role of TPH on hepatic stellate cells may have an important implication in HCV mediated liver disease progression.

Immortalized human stellate cells LX2 (kindly provided by Scott Friedman, Mount Sinai School of Medicine, NY), when cultured on Matrigel coated plates in SABM (Clonetics, CA) supplemented with glutamine (2× concentration) and 0.2% BSA, formed extensive network like structure. LX2 cells growing on Matrigel, when incubated with conditioned culture medium from IH (conditioned medium-CM) or THLE (primary human hepatocytes immortalized by SV 40 T antigen-kindly provided by Curtiss C. Harris, NCI) stably transfected with HCV core gene, became granular and cell death occurred within 6 days. However, cell death was not observed when LX2 cells were incubated with CM from THLE as a negative control.

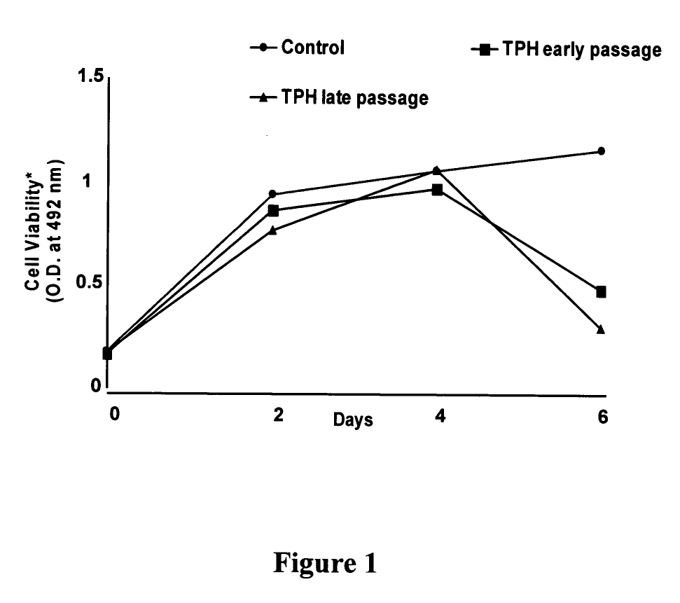

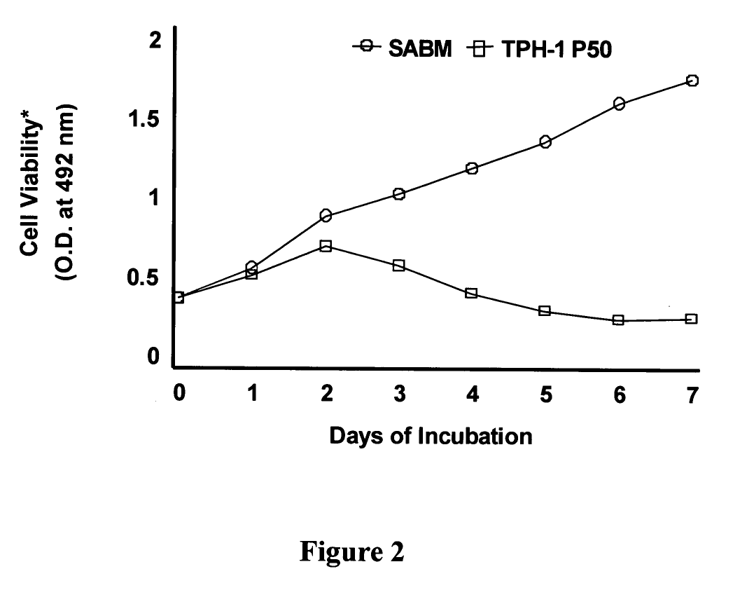

LX2 cells plated on a plastic surface grew as activated stellate cells. Upon incubation with CM from TPH or THLE transfected with HCV core gene, LX2 cells became granular after three days and cell death was observed (FIG. 1). We also examined the role of CM from human hepatocytes on rat stellate cells. Primary rat stellate cells (kindly provided by Bruce Bacon, Division of Gastroenterology, Saint Louis University) growing on Matrigel coated plate displayed cell death upon incubation with CM from TPH or THLE-core (FIG. 2). On the other hand, CM from THLE (negative control) displayed slightly higher cell growth. These preliminary results suggested that conditioned medium from HCV core transfected hepatocytes causes both human and rat stellate cell death.

Immortalized hepatocytes (TPH) displayed preferential growth when cocultured with activated hepatic stellate (LX2) cells. Further studies suggested that LX2 cells die when grown with TPH in dual chambers or incubated with conditioned medium from IH even at a 1:64 dilution. However, LX2 cell death was not observed when incubated with conditioned media from hepatocytes or from non-hepatic epithelial cells (HeLa, BHK, or MCF-7), indicating that TPH secrete a specific death factor for LX2 cells. Further analysis indicated the active factor responsible for HSC death was fragmented gelsolin. Stellate cell cytotoxicity by conditioned medium from TPH may have important implications in HCV mediated liver disease progression.

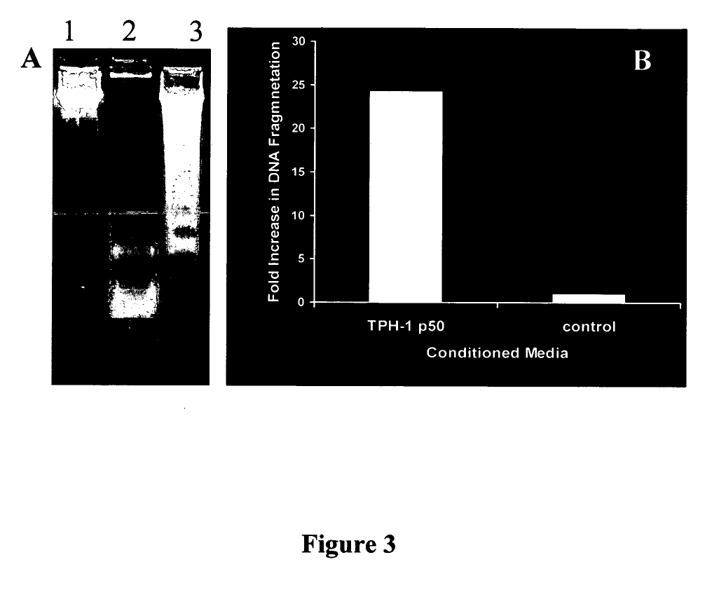

To determine how stellate cell death occurs, cells were harvested on day 6 and examined for characteristic DNA ladder of apoptosis. DNA from rat and human HSC incubated with the CM from TPH or THLE-core displayed apoptotic signature oligonucleosome fragments by agarose gel electrophoresis (FIG. 3, panel A). The level of apoptosis in LX2 cells was also quantified by a cell death detection ELISA, which is based on the quantitative sandwich-immunoassay principle using mouse monoclonal antibodies against DNA and histone. This allows for the specific determination of oligonucleosomes in the cytoplasmic fraction of the apoptotic cells. Analysis of the LX2 cells incubated with the CM from TPH or THLE-core suggested a significant level of apoptotic cell death as compared to the LX2 cells incubated in normal medium or conditioned medium from negative control THLE cells (FIG. 3, panel B).

### EXAMPLE 2

### Methods of Immortalizing Hepatocytes

Methods of producing immortalized hepatocytes for use in producing the conditioned media of the instant invention are described in detail in Ray et al., 2000, and Basu et al., 2002, which are herein incorporated in their entirety by reference. An exemplary method is summarized below.

Cell growth regulatory potential of HCV core protein was investigated by introduction of the core genomic region into primary human hepatocytes, a natural host for virus replication and tropism (Ray et al., 2000). Interestingly, core transfected primary human hepatocytes (TPH) were immortalized and exhibited continuous growth for more than three years. In contrast, similar transfection with core deletion mutants (Core aa 26-85 and Core aa 80-150) or gene encoding nucleocapsid protein (NP) from an unrelated human parainfluenza type 3 virus (HPIV-3) as controls did not immortalize primary human hepatocytes. We have so far established immortalized hepatocytes from 3 different healthy donors and cells from another donor became contaminated by yeast and we could not recover cells from that culture.

Core transfected immortalized hepatocytes exhibited HCV core protein expression, albumin secretion, glucose phosphatase activity, and absence of smooth muscle actin (Ray et al., 2000). Cells in culture displayed focal cytoplasmic and membrane staining with a polyclonal anti-CEA (Dako rabbit anti-human CEA, A 0015), which has specificity for a range of related cell adhesion glycoproteins including carcinoembryonic antigen (CEA), biliary glycoprotein (BGP1/CEACAM1), and nonspecific cross reacting antigen (NCA/CEACAM6).

RNA extracted from the immortalized hepatocytes was examined for hepatobiliary transport marker genes. Three sets of sense and antisense oligonucleotide primers (Zollner et at, 2001) were used for detection of mRNA of multidrug resistance-associated protein (MRP), liver-specific organic anion transporter (LST1), and human Na+-taurocholate cotransporting polypeptide (NTCP). Primer sequences were selected from the respective cDNA sequences submitted in the GenBank (accession numbers ABOIO887, AFO60500 and L21893).

| | |
|---|---|
| MRP-2 sense primer: | |
| CACCTTAGTGCAGCGCTTCTA | (SEQ ID NO: 2) |
| MRP-2 antisense primer: | |
| AGGTCTCTCAGCACCAGGTCTAGG | (SEQ ID NO: 3) |
| NTCP sense primer: | |
| AACGCGTCTGCCCCATTCAAC | (SEQ ID NO: 4) |
| NTCP antisense primer: | |
| GACGGCCACACTGCACAAGAGA | (SEQ ID NO: 5) |
| LST-1 sense primer: | |
| GAAGATGTTCTTGGCAGCTCT | (SEQ ID NO: 6) |
| LST-1 antisense primer: | |
| GATCCCAGGGTAAAGCCAAT | (SEQ ID NO: 7) |

Identical quantities of RNA were subjected to RT-PCR (BRL) using specific primers for amplification (˜600 bp long). The amplified DNAs were subjected to gel electrophoresis. The relative abundance of MRP-2 and LST-1 was significantly higher than NTCP in immortalized cells under our experimental conditions. RNA from human foreskin fibroblasts was used as a negative control in this experiment and did not exhibit amplification of specific bands. Results from this set of experiments further suggested the presence of hepatocyte specific markers in the immortalized cells.

An enhancement of telomere length, a characteristic of immortalized or transformed cells, was evident upon passage of the immortalized hepatocytes (Ray et al., 2000). Results from these studies suggested that HCV core protein promotes immortalization of primary human hepatocytes, which may predispose cells for transformation.

We also examined whether suppression of core genomic sequence has an effect upon the maintenance of immortalized hepatocytes and if there are any corresponding consequences on cellular gene expression. Results from these studies suggested that antisense RNA-mediated reduction of core protein function, at an early stage after hepatocyte immortalization, results in cell death. This might occur by regulation of cell cycle related genes, possibly by elevating p53 expression level (Basu et al., 2002). These results further demonstrated that hepatocyte immortalization is not due to an artifact of spontaneous clonal selection. However, antisense core gene expression did not exhibit apoptotic cell death in immortalized hepatocytes from late passage.

### EXAMPLE 3

### Induction of TRAIL-Mediated Apoptosis

Activated HSCs are central to the pathogenesis of liver fibrosis/cirrhosis, both as a source of fibrillar collagens that characterize fibrosis/cirrhosis and tissue inhibitors of matrix degrading metalloproteinases (TIMPs). Moreover, activated HSC apoptosis plays a critical role in the spontaneous recovery from biliary fibrosis (Issa et al; 2001). Both survival and apoptosis of HSC are regulated by growth factors expressed during fibrotic liver injury. We have previously shown that HCV core protein mediates immortalization of primary human hepatocytes, a natural host for virus replication and tropism (Ray et al 2000). In this study, we investigated the relationship between HCV core protein mediated immortalized human hepatocytes (IH) and activated HSC. To study the relationship between the IH and activated HSCs, we used a spontaneously immortalized human stellate cell line (LX2) and primary rat HSCs. These two different cells were co-cultured and examined for cell growth. IH preferentially grew and suppressed proliferation of activated LX2 cells. The number of LX2 cells decreased by >90% within 96 hours. The LX2 cells and IH were identified by immunofluorescence using anti-SMA antibody, and a hepatocyte specific monoclonal antibody. The suppression of activated stellate cell growth could be due to a higher growth rate of the IH or due to the regulation of activated HSCs by the immortalized hepatocytes either through a receptor interacting protein-dependent mechanism, or by secretion of a soluble mediator.

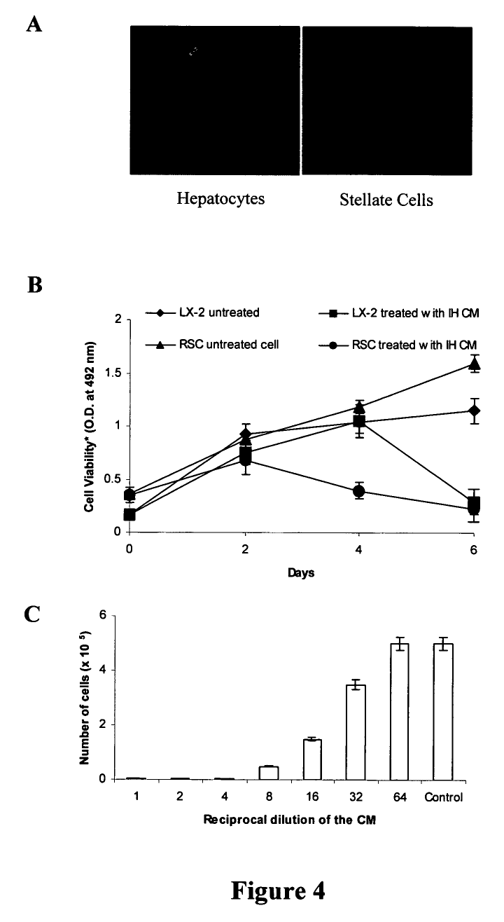

We have previously investigated the cell growth regulatory potential of HCV core protein by introduction of the core genomic region into primary human hepatocytes, a natural host for virus replication and tropism (Ray et al 2000). During that study, at −6-8 weeks after transfection, hepatocytes exhibited a shift from senescence to a replicative stage. The growth of the hepatocytes were examined by immunofluorescence using a hepatocyte specific monoclonal antibody Hep Par (FIG. 4, panel A). Primary hepatocyte preparations generally contain a small percentage of contaminating stellate cells. Activated hepatic stellate cells (HSC) were also observed in the transfected hepatocyte culture by immunofluorescence, and were observed to be present 6-8 weeks after transfection using an antibody against smooth muscle actin (SMA), a marker for activated stellate cells (FIG. 4, panel A). Interestingly, when core transfected primary hepatocytes entered from senescence to replicative stage, they preferentially grew, and replaced the activated HSCs, which could not be detected within 4 weeks of this shift.

To further investigate whether the suppression of activated LX2 cell proliferation by IH was through a receptor interacting mechanism or through a soluble mediator, we cultured the LX2 and IH on either side of a dual chamber in a Transwell dish separated by a 0.45 μm filter. IH suppressed the proliferation of the activated LX2 cells indicating that IH might be secreting a soluble mediator into the culture medium to suppress LX2 cell proliferation. To further verify our result, we incubated LX2 cells with conditioned medium (CM) from the IH. LX2 or rat HSCs became granular upon incubation with CM, and complete disruption of the cell monolayer with suppression of cell proliferation was observed between 2-4 days of incubation (FIG. 4 panel B). These results indicated that the soluble mediator in culture medium from IH may not be species specific for stellate cells. The activity of CM on LX2 growth control proportionately decreased with increasing dilutions of the CM. (FIG. 4, panel C). We also examined the viability of HSC from disrupted monolayer by trypan blue dye exclusion. Majority of the disrupted cells from monolayer (>90%) retained trypan blue stain indicating cell death. We examined the role of CM on hepatic (Huh-7, and THLE) and non-hepatic (HeLa, MCF-7, and BHK) cell growth. Growth suppression was not observed with any one of these cell lines, indicating that soluble mediator from IH acts specifically on hepatic stellate cells. Together, our results indicated that IH secrete a soluble mediator that causes LX2 cell death.

To investigate whether other immortalized cell types secrete death factor, we incubated LX2 cells with the CM from non-hepatic (HeLa, MCF-7, BHK, CHO) and hepatic (HepG2, Hep3B, Huh-7 and THLE) cell lines. LX2 growth suppression was not observed with CM from non-hepatic and two of the hepatic cell lines (THLE and Huh-7). However, CM from HepG2 or Hep3B cells induced LX2 cell growth suppression death in a manner similar to IH. Both HepG2 and Hep3B are transformed human hepatocytes. These observations indicated that the soluble mediator released in conditioned medium, was not limited to some of the transformed hepatocytes, and is not due solely to the presence of HCV core protein.

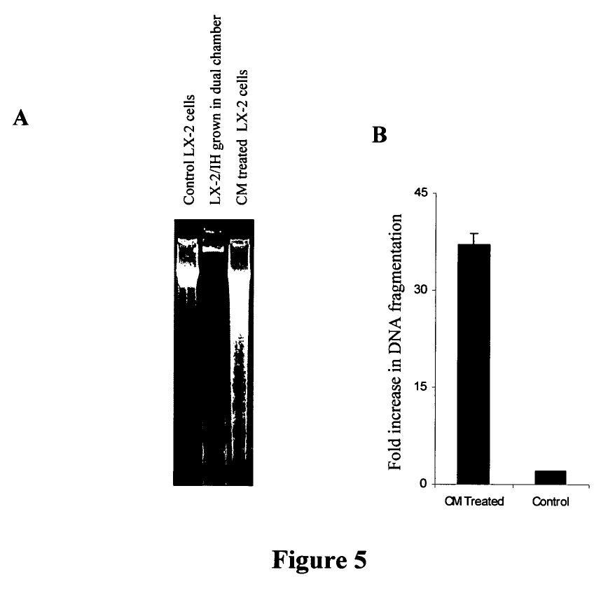

To determine whether the observed LX2 cell death was associated with apoptosis, LX2 cells were incubated with the CM from IH or cocultured with IH in a dual chamber. LX2 cells harvested after 4 days of incubation with IH, displayed apoptotic signature oligonucleosome fragments by agarose gel electrophoresis (FIG. 5 panel A). DNA fragmentation of LX2 cells was quantified by a cell death detection ELISA, which is based on the quantitative sandwich-immunoassay principle using mouse monoclonal antibodies against DNA and histone. This allows for the specific determination of oligonucleosomes in the cytoplasmic fraction of the apoptotic cells. ELISA with LX2 cells, prior incubated with the CM from IH or cocultured with IH in dual chambers, suggested a significant increase (40 fold) of apoptotic cell death as compared to LX2 cells incubated with culture medium (FIG. 5 panel B).

To identify the soluble mediator, we compared the cytokine expression profile of the CM from IH and THLE (which does not cause LX2 apoptosis) using multiple human cytokine array. An increase of ˜10 fold in TIMP-1, ˜4 fold of TIMP-2, and ˜2 fold each of FGF-9, IGFBP-4, and osteoprotegrin levels were observed in the CM from IH (Table 2). On the other hand, the levels of interleukins and TNF related cytokines (TGF-β, TNF-α TNF-β, and IGF-1) remained similar in the CM of IH and THLE, indicating that these cytokines are not responsible for LX2 cell apoptosis (Table 2). We have observed that the activity of the soluble modulator from IH causing LX2 cell death is lost upon incubation at 56° C. for 5 minutes.

TABLE 2
Cytokine profile of the CM from IH relative to CM from THLE cells

| Functional gene grouping | Fold change |
|---|---|
| Interleukins | |
| IL-1, IL-2, IL-3, IL-4, IL-5, IL-6, IL-7, IL-8, | No change |
| IL-10 | |
| IL-12, IL-16 | |
| TNF related cytokines | |
| TGF-β, TNF-α, TNF-β, IGF-I | No change |
| EGF/FGF | |
| EGF, FGF-4, FGF-6, FGF-7 | No Change |
| FGF-9 | +2 |
| TIMP Family | |
| TIMP-1 | +10 |
| TIMP-2 | +4 |
| IGFBP Family | |
| IGFBP-1, IGFBP-2, IGFBP-3 | No change |
| IGFBP-4 | +2 |
| Others | |
| Osteoprotegrin | +2 |
| RANTES | No change |

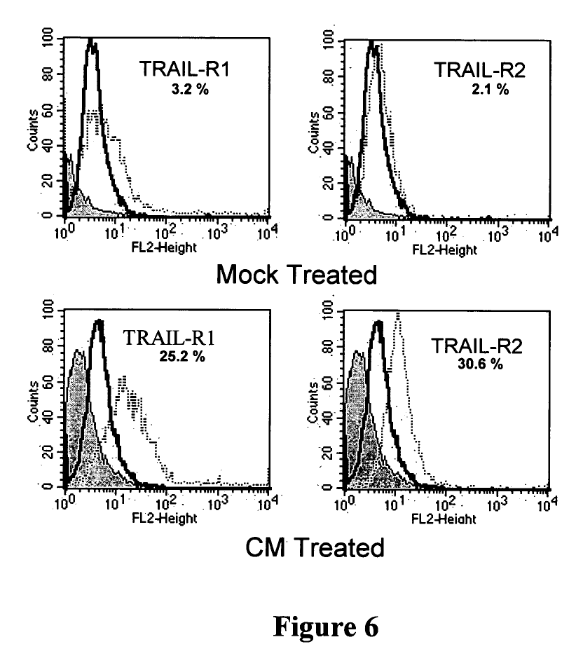

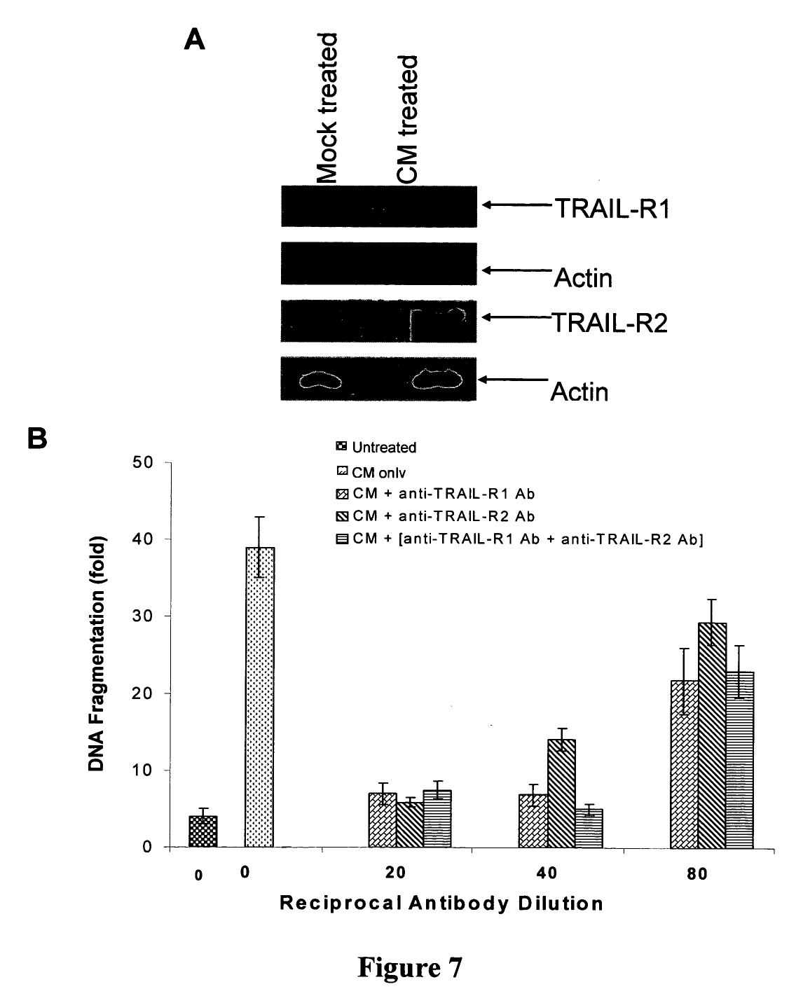

Among the extrinsic apoptotic pathways, FAS and TNF-α were not utilized by the soluble mediator to induce apoptosis in LX2 cell since addition of anti-Fas Ab or TNF-α did not induced apoptosis of LX2 cells. However, the addition of histidine tagged rhTRAIL along with anti-polyhistidine antibody induced apoptosis of LX2 cells. Therefore, we examined whether the soluble mediator utilizes the TRAIL pathway to induce apoptosis. TRAIL induced apoptosis can often lead to an increase in expression of TRAIL receptors (Zhang et al. 1999; Wang and E1-Diery, Oncogene, 22, 8628). We first examined whether the TRAIL receptors are modulated in LX2 cells upon incubation with CM and observed an upregulation of DR4 (TRAIL-R1) and DR5 (TRAIL-R2) expression. FACS (FIG. 6) and Western blot (FIG. 7, panel A) analyses of CM treated LX2 cells suggested an elevation of TRAIL-R1 and TRAIL-R2 expression, as compared to the media control. Densitometric scanning suggested an ˜4 fold increase in the individual expression of DR4 and DR5 (FIG. 7, panel A). These findings suggested that the soluble mediator in CM may be targeting TRAIL pathway to induced apoptosis in LX2 cells. Addition of a polyclonal antibody to DR4 or DR5 inhibited CM induced apoptosis of LX2 cells in a dose dependent manner. Each antibody inhibited CM induced apoptosis at 10 ug/ml of IgG (FIG. 7, panel B). However, inhibition of cell death was not augmented by the presence of both the antibodies. Interestingly, treatment of CM with commercially available neutralizing antibody to hTRAIL (20 or 40-ng/ml) did not inhibit LX2 apoptosis, although same or lower concentration of the neutralizing antibody inhibited rhTRAIL induced apoptosis of LX2 cells. These findings suggested that the soluble mediator from IH utilizes TRAIL signaling pathway to induce apoptosis of LX2 cells. Similar observations were also reported by Fisher et al. (2002), during stellate cell killing by activated Kupffer cells. Activated Kupffer cells induce apoptosis of HSCs through upregulation of the DR4 and DR5 receptors, although the addition of the neutralizing antibodies to TRAIL did not inhibit HSC apoptosis. Thus, the soluble mediator in the CM of IH induces apoptosis of LX2 cells by utilizing TRAIL receptors.

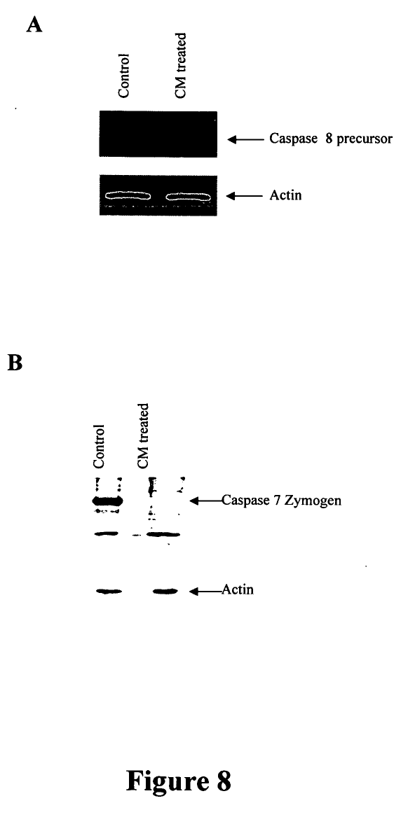

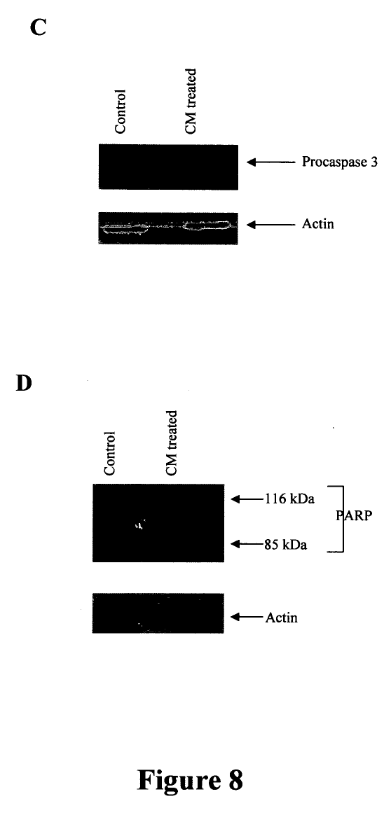

To further investigate the apoptotic signaling pathway we analyzed caspase activation and PARP cleavage in CM treated LX2 cells by Western blot. Decrease in the expression of procaspase 8 in CM treated LX2 cells as compared to untreated cells suggested the activation of procaspase 8 in CM treated LX2 cells (FIG. 8, panel A). However, activation of caspase 9 was not observed in the control or CM treated cells. Caspases 3, 7 and PARP play a key role in the final or execution phase of apoptosis. The cell lysates were similarly subjected to Western blot analysis for detection of caspases 3, 7, and PARP cleavage. Activation of caspase 7 (FIG. 8, panel B), not caspase 3 (FIG. 8, panel C) was observed. Furthermore, cleavage of the DNA repair enzyme PARP was similarly examined. The 116 kDa polypeptide was cleaved to ˜86 kDa signature peptide upon treatment of LX2 cells with CM (FIG. 8, panel D).

A soluble mediator secreted from IH induces apoptosis in HSC. Our observations suggested the involvement of TRAIL-R1 and TRAIL-R2 receptors in CM mediated apoptosis of LX2 cells via the caspase-8 apoptotic pathway. The apoptosis inducing soluble mediator from serum free CM of IH was purified and identified as described in Example 4.

### EXAMPLE 4

### Purification and Identification of Stellate Cell Death Factor

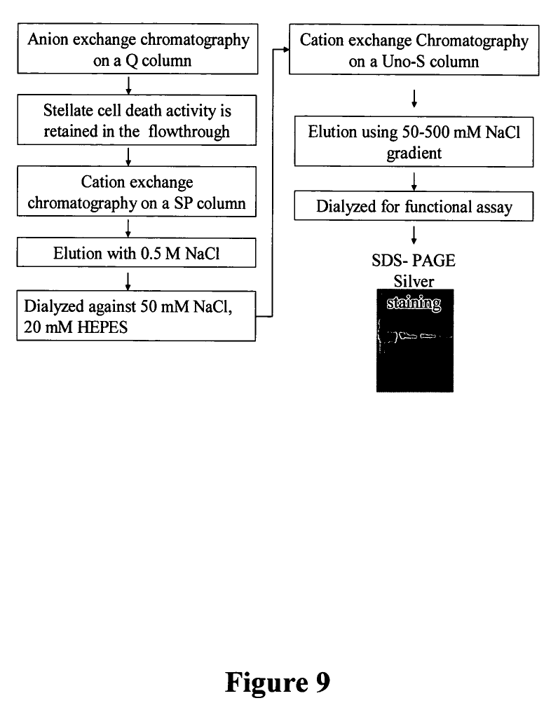

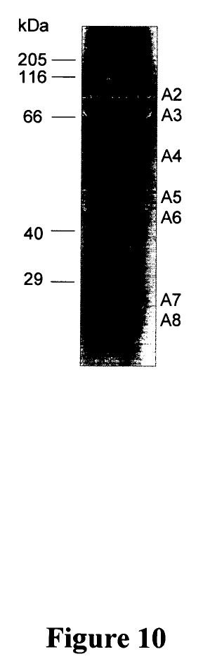

In order to identify the soluble mediator for stellate cell cytotoxicity, CM was subjected to a two step cation- and anion-exchange chromatography (FIG. 9). The cytotoxic activity of the purified material was monitored at each step of purification. Media conditioned by immortalized hepatocytes (supra) were concentrated, then diluted with four volumes of buffer H (20 mM Hepes, pH 7.4, 15% glycerol), and loaded onto a 2 ml Q-Sepharose column that had been pre-equilibrated with buffer H. The flow through from the void volume of the column exhibited stellate cell death. The active fraction was subsequently loaded directed onto a 2 ml SP-column. After washing the column with 5 ml of buffer H, the bound protein was eluted with 5 ml of buffer H containing 0.5 M NaCl. (FIG. 4) Fractions (1 ml) were collected and evaluated for LX2 cell death. The active fraction was analyzed by SDS-PAGE (FIG. 10). Individual protein bands A2-A8 were cut from the gel and analyzed by peptide mass fingerprinting (LC-MS). Amino acid sequence homology with NCBI database indicated: A2 and A3 contain human albumin, A4 contains ezrin and bands A5-A8 contain 50, 46, 25, and 23 kDa fragments of gelsolin respectively (Table 3). Fingerprints were searched with the program MS-FIT (prospector.ucsf.edu/ucsffitml/msfit.htm) using all human cellular proteins in the NCBI database.

TABLE 3
Target proteins of interest identified by LC-MS

| | | | | Molecular | |
|---|---|---|---|---|---|
| | | | | weight | |
| | | | | (′˜kDa)of | |
| | | | | the | |
| | | Molecular | | polypeptide | |
| Target | Accession | Weight | | bands | Amino acid sequences |
| Protein | Number | (kDa) | Function | analyzed | and locations |
| Ezrin | giI21614499 | 69.3 | Belongs to the | 64 kDa | LFFLQVK (residues 101-107) |
| | | | family of | | and IGF PWSEIR |
| | | | actin-binding | | (residues 238-246) |
| | | | proteins and | | |
| | | | act both as | | |
| | | | linkers | | |
| | | | between the | | |
| | | | actin | | |
| | | | cytoskeleton | | |
| | | | and plasma | | |
| | | | membrane | | |
| | | | proteins and | | |
| | | | as signal | | |
| | | | transducers in | | |
| | | | responses | | |
| | | | involving | | |
| | | | cytoskeletal | | |
| | | | remodelling | | |
| Gelsolin | giI4504165 | 85.6 | Regulation of | 50 kDa | KAGKEPGLQIWR(residues |
| | | | actin | | 61-72) and |
| | | | dynamics, Fc | | QTQVSVLPEGGETPLFK |
| | | | and integrin | | (residues 374-390). |
| | | | mediated | 46 kDa | AGKEPGLQIW |
| | | | phagocytosis, | | (residues 62-73), IFVWK |
| | | | apoptosis | | (residues 342-346), and |
| | | | | | QTQVSVLPEGGETPLFK |
| | | | | | (residues 374-390) |
| | | | | 25 kDa | IFVWK (residues 342-346), |
| | | | | | and QTQVSVLPEGGETPLFK |
| | | | | | (residues 374-390) |
| | | | | 23 kDa | QTQVSVLPEGGETPLFK |
| | | | | | (residues |
| | | | | | 374-390) and AGALNSNDAFVLK |
| | | | | | (residues 585-597) |

The proteins identified by LC-MS suggested the necessity for examination of the functional activities of gelsolin and ezrin on LX2 cells. Other proteins identified are known to lack apoptotic activity as an external stimuli, or represented components of cell culture medium. To examine the role of ezrin for cytotoxic activity, a monoclonal antibody (Clone 3C12, IgG1, Zymed, CA) and rabbit antiserum directed against a synthetic peptide corresponding to the residues surrounding Thr567 of human ezrin (Cell Signaling, CA) were used separately to adsorb out ezrin from CM. For this, the antibodies were immobilized on Protein G Sepharose (Amersham, N.J.), and CM was incubated with the immobilized antibody on Protein G. The suspension was centrifuged to separate the beads and clear CM was filtered through hydrophilic Durapore (PVDF) membrane (Millipore Corp) for sterilization. Ezrin depleted CM and mock treated control CM were added to LX2 cells and incubated for evaluation of apoptosis. Results suggested that adsorption of CM with anti-ezrin antibody display LX2 toxicity, similar to mock treated CM control. The same results were obtained when CM was preincubated with both antibodies.

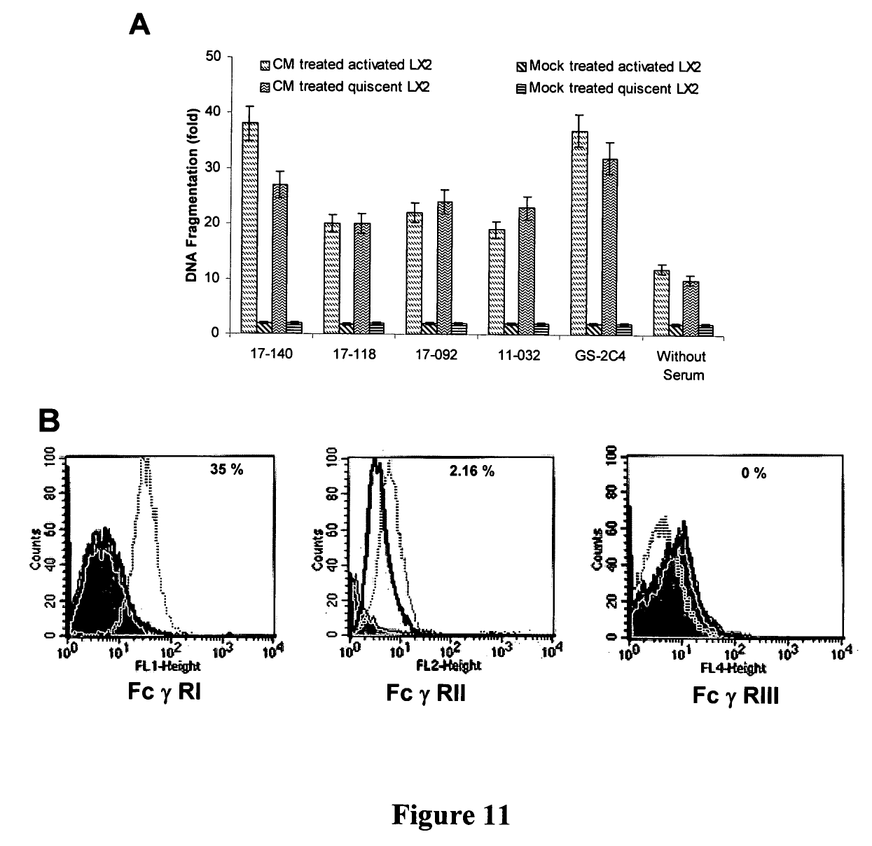

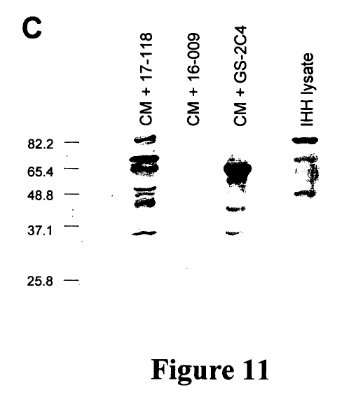

Similarly, two different murine monoclonal antibodies to gelsolin GS-2C4, IgG1 isotype, (Sigma) and Clone 2, IgG2a subclass (BD Biosciences) were incubated with the CM. The monoclonal antibody GS-2C4 is directed to an epitope located on the 47 kDa peptide derived from a chymotryptic cleavage of human plasma gelsolin, reacts with plasma and cytoplasmic gelsolin, and recognizes an epitope containing the carboxy terminal actin binding site. The other monoclonal antibody, Clone 2, is directed between amino acid residues 592-768 fragment of gelsolin. Interestingly, when CM preincubated with GS-2C4 markedly increase the cytotoxic activity of CM even up to a dilution of 1/80 of the mouse ascites by reducing the time of incubation from 72 h to less than 24 h (FIG. 11A), Purified mAb from Clone 2 did not enhance or inhibit the cytotoxic effect of CM treated LX2 cells. A reverse titration CM displayed cytotoxic activity only up to ¼ dilution at a fixed GS-2C4 concentration (at 1/20 dilution of mouse ascites). This observation suggested that the concentration of active component in CM for LX2 toxicity is low. Earlier studies suggest that N-terminal gelsolin fragment (1-352), which contains the severing activity, but not of the COOH-terminal fragment (353-731), triggered rapid depolymerisation of the actin cytoskeleton (Kothaka et al., 1997).

We do not know at this time whether gelsolin is cleaved intracellularly or proteolytically degraded in the extracellular environment. Interestingly, purified full-length plasma gelsolin (5 ug/ml) when added to LX2 did not induce cell death. Future study should help in further understanding the role of specific fragment of gelsolin in mediating apoptosis, and inhibition of apoptotic activity by suitable antibodies.

### EXAMPLE 5

### Enhanced Cytotoxic Through Antibody Binding

The inventors have made the surprising discovery that when CM was preincubated with antibody directed against gelsolin, there was a marked increase in cytotoxic activity. To examine the role of gelsolin, two different murine monoclonal antibodies (clone GS-2C4, IgG1 isotype, Sigma; and Clone 2, IgG2a isotype, BD Biosciences), were used to determine the altered function of CM in inducing LX2 apoptosis. The monoclonal antibody GS-2C4 recognizes an epitope containing carboxy terminal actin binding site located on a 47 kDa peptide derived from a chymotryptic cleavage of human plasma gelsolin. This antibody does not immunoprecipitate 93 kDa full-length gelsolin, but recognizes in Western blot analysis. The other monoclonal antibody, Clone 2, is directed between amino acid residues 592-768 of gelsolin. Interestingly, when CM was preincubated with GS-2C4, a marked antibody-dependent enhancement of LX2 cell death was observed within 24 h of incubation (FIG. 11A). In contrast, purified monoclonal antibody from Clone 2 did not enhance or inhibit the cytotoxic effect of CM upon LX2 cells.

To examine whether autoantibody is generated against gelsolin in chronically infected HCV patient sera, CM was incubated with sera from 12 patients before addition to LX2 cells for apoptosis. Sera from 6 healthy subjects were used as negative controls. Enhancement of LX2 apoptosis was observed in 4 of 12 patient sera within 24 h of incubation (FIG. 11A). Pre-treatment of these chronically infected patient sera with antibody to the heavy chain of human IgG inhibited LX2 cell death, suggesting autoantibodies of the IgG subtype are generated against gelsolin fragments, and that they play a role in the augmentation of programmed cell death in stellate cells. These results also suggested that gelsoln fragments are generated in vivo, and induce auto-antibodies in patient sera. FACS analysis suggested that LX2 cells predominantly express FcyRI (CD64) on cell surface (FIG. 11B). Since the Ig-binding subunit of FcyRI has the ability to bind certain subtypes of IgG (IgG1 and IgG3) with a high affinity, our results suggest the generation of IgG1 or IgG3 autoantibodies against gelsolin in chronically infected HCV patients. We examined the interaction of patient sera with CM by immunoprecipitation, followed by Western blot analysis using monoclonal antibody (GS-2C4) to gelsolin. In addition to full-length gelsolin, a series of low molecular weight fragments of gelsolin (37-70 kDa) was observed in the blot (FIG. 11C). Western blot analysis using IH extracts also suggested the presence of gelsolin fragments, implicating intracellular generation of fragmented gelsolin which are secreted into the CM. Similar fragmentation of gelsolin was also observed from analysis of CM at different time points of IH culture (data not shown). Interestingly, purified full-length plasma gelsolin (5 ug/ml) when added to LX2 cell culture did not induce cell death. Taken together, our results suggested that fragmented gelsolin induces apoptosis of LX2 cells, which is augmented by IgG1 antibody for binding through CD64 receptor on cell surface. This specificity towards fragmented gelsolin was not seen in a panel of sera from healthy individuals. Antibody response to this self modified protein antigen has not previously been recognized or evaluated. These results suggest that a mechanism which enhances autoimmune recognition of gelsolin, possibly by antigen unmasking through fragmentation, may result in increased cytotoxic activity through binding of high affinity Fc receptors.

### Materials and Methods

HCV core immortalized human hepatocytes (1H) were generated by transfection of primary human hepatocytes with the plasmid DNA expressing core genomic region of genotype 1a (Ray et al, 2000). Transfected hepatocytes were seeded on a collagen type I coated plate and maintained at 37° C. in a defined culture medium supplemented with growth factors and antimicrobial agents (SAGM, Clonetics, Walkersville Md.), or with DMEM supplemented with 5% FBS.

A spontaneously immortalized human stellate cell line (LX2) was kindly provided by Dr. Scott L. Friedman (Mount Sinai School of Medicine, NY). LX2 cells are a low-passaged human cell line derived from normal human stellate cells that are spontaneously immortalized. These cells were selected by their ability to grow under low serum conditions (1% fetal bovine serum) and express α-SMA under all culture conditions (Taimr, P. Hepatology, January 2003, Volume 37, Number 1). LX2 cells were grown in activated state on plastic dishes, in Dulbecco's minimum essential medium PMEM; BioWhittaker, Walkersville, Md.) supplemented with 5% fetal bovine serum, 100 U/mL penicillin, 100 μg/mL streptomycin, 2× L-glutamine. LX2 cells also grew in defined culture medium for immortalized hepatocytes when supplemented with 2× glutamine. Primary stellate cells from rat liver (kindly provided from the laboratory of Dr. Bruce Bacon, Saint Louis University) were grown in Dulbecco's medium supplemented with 10% fetal bovine serum.

Monoclonal and polyclonal antibodies to caspases 3, 7, hTRAIL and poly histidine were obtained from R&D Systems (Mineapolis, Minn.), caspase 9, Fas, Fas-L were obtained from Pharmingen (San Diego, Calif.), while antibodies to PARP, DR4, DR5 and caspases 8 were obtained from Alexis Biochemicals (Carlsbad, Calif.). rhTNF-α and recombinant hTRAIL were obtained from Promega and R&D Systems.

LX2 and IH were cocultured for 3 days under conditions permitting either cell-to-cell contact or in transwell chambers. The ratio of IH to LX2 at the onset of culture was 1:1. For coculture, LX2 and IH cells were grown for 2-4 days in SAGM (Clonetics Walkersville, Md.) supplemented with 2× glutamine and 5% chemically denatured serum (BioSource, MD). Cocultures were also performed in Transwell dual chambers (Costar). The two compartments were separated by a porous polycarbonate membrane (0.45 μm pore diameter), which allows free exchange of soluble factors between the two compartments. In transwell chambers, IH cells were seeded in the upper compartment while the bottom compartment contained LX2 cells.

LX2 and IH were identified by immunofluorescence using activated HSC specific anti-smooth muscle actin antibody (Sigma, St. Louis, Mo.) and hepatocytes specific monoclonal antibody (DAKO, Carpinterin, Calif.). Briefly, cells were grown on cover slips, washed with PBS, and fixed with 10% formaldehyde for 15 minutes at room temperature. Fixed cells were incubated for 1 h with a mouse monoclonal antibody to α-smooth muscle actin or hepatocyte specific antibody at appropriate dilutions. Cells were extensively washed and incubated for 1 h with FITC-conjugated anti-mouse IgG. Control cells were processed similarly without incubation with the first antibody. Cover slips were mounted in anti-fade reagent, and the cells were observed using a fluorescence microscope.

IH were grown on a collagen type 1 coated plate in SAGM supplemented with 5% chemically denatured serum at 37° C. At ˜80-90% confluency, cells were washed extensively and incubated with serum free SAGM. The culture medium was collected as conditioned medium (CM) from IH after 48 hours. CM was clarified by centrifugation at 6,000 g to remove cell debris, supplemented with 2×L-glutamine, and aliquoted for storage at −20° C. until use. Conditioned medium from HeLa, MCF-7, BHK, CHO, HepG2, Hep3B and Huh-7 cells, maintained in Dulbecco's essential medium supplemented with 10% fetal calf serum, were prepared in a similar manner.

LX2 and rat HSC proliferation were assessed using a CellTiter 96 Aqueous non-radioactive cell proliferation assay (Promega, Madison, Wis.). This assay is composed of a novel tetrazolium compound [3-(4,5-dimethylthiazol-2-yl)-5-(3-carboxy methoxyphenyl)-2-(4-sulphophenyl)-2H-tetrazolium, inner salt, MTS] and an electron coupling reagent (phenazine methosulfate; PMS). MTS is bioreduced by cells into formazan, that is soluble in cell culture medium. The conversion of MTS into aqueous, soluble formazan is accomplished by a dehydrogenase enzyme found in metabolically active cells. Thus, the quantity of formazan produced in cell culture medium is directly proportional to the number of living cells. After 2 days of culture in a 96 well plate, LX2 cells were incubated with CM from IH. LX2 cells were harvested at different time points and their growth were compared with cells grown in serum free SAGM, supplemented with 2× L-glutamine.

IH, HepG2 and Huh-7 cells were grown in 35-mm plates to −90% confluency. Cell monolayers were washed with medium lacking methionine and cysteine and incubated in the same medium for an additional 30 min. The cells were then incubated in medium containing 50 μCi/ml of ³⁵S-protein labeling mix (Amersham) for 18 hours. Cell culture supernatant was collected after 24 h, centrifuged to remove cell debris and was concentrated using a membrane filter with exclusion limit >50 kDa proteins (Millipore, Bedford, Mass.). The concentrated supernatant was mixed with equal amounts of sample buffer (2×) and analyzed by 8.5% SDS-PAGE.

RayBio™ human cytokine array (RayBio™, Atlanta, Ga.) was used to identify the expression profile of multiple cytokines following the manufacturer's procedure. Briefly, CM from IH and a different human hepatocyte cell line (THLE, immortalized by SV40 T antigen kindly provided by Curtis C. Harris, NCI) were concentrated and incubated with the protein array membrane containing antibody against the cytokines. Following incubation, the membrane was washed and developed by the addition of horse radish peroxidase conjugated streptavidin and substrate, followed by chemiluminescence. The image from the membrane exposed to X-ray film was scanned to quantitate cytokine levels using a densitometric scanner after normalizing with the controls.

Western blot analysis was performed to analyze the expression level of DR4, DR5, caspases 3, 7, 8 and 9 using specific antibodies in control and experimental cells. Briefly, equal amounts of whole cell lysates in sample buffer were separated by SDS-PAGE, and transferred onto nitrocellulose membrane. The separated proteins were incubated with specific antibody, followed by a HRP conjugated secondary antibody, and detected by chemiluminescence. Cellular actin was detected similarly in a reprobed blot for use as an internal control for relative quantitation of the proteins in control and experimental cells by densitometric scanning.

Fluorescence-activated-cell-sorter (FACS) analysis: LX2 cells (either untreated or treated with CM) were treated with anti-TRAILR1 (DR4), anti-TRAILR2 (DR5), FcyR1, FcyR2, FcyR3 or isotype specific antibodies (negative control) antibodies conjugated to different fluorochromes (FITC, PE, and Alexa 647) for FACS analysis. Nonspecific background was determined from untreated and isotype matched unrelated negative control antibodies. Positive cells were detected by FACScan (Becton Dikinson) and results were analyzed with Cell Quest Version 3.2 software. Tenthousand cells were analyzed for each sample and a gate was set on the basis of a dot plot for 90° light scatter versus forward angle light scatter to exclude dead cells and debris from analysis.

The concentrated CM (˜20 fold) was first diluted with four volumes of buffer H (20 mM Hepes, pH 7.4, 15% glycerol), and loaded onto a 2 ml Q-sepharose column that was pre equilibrated with buffer H. The flow through from the void volume of the column exhibited stellate cell cytotoxicity. This active fraction was subsequently loaded onto a 2 ml SP-column. After washing the column, bound protein was eluted with 5 ml of buffer H containing 0.5 M NaCl. Fractions (1 ml) were collected and evaluated for LX2 cell death assay, and protein in each fraction was analyzed by SDS-PAGE, followed by silver staining. Active fractions eluted from the SP-column were pooled, dialyzed against buffer H (3×500 ml), and loaded on to UNO-S FPLC column. The bound protein was eluted with a liner gradient of 0 to 0.5M NaCl in 20 ml of buffer H with a flow rate of 1 ml/min. Each fraction was analyzed for LX2 cell death, as well as by SDS-PAGE and silver staining. Protein bands were cut from the gel, digested with trypsin, and identified by peptide mass fingerprinting. LC-MS fingerprints were searched with the program MS-FIT (prospector.ucsf.edu/ucsfhtml/msfit.htm) using all human cellular proteins in the NCBI database. The N-terminal amino acid sequencing was done by Midwest Analytical, Inc (St. Louis, Mo.) following Edman degradation.

MALDI-TOF/MS analysis of the purified soluble mediator was performed with a (Voyager DEPRO Perseptive) MALDI mass spectrometer. In brief, protein samples were solubilized for 30 min at ambient temperature in 9 M urea, 1% CHAPS, 70 mM dithiothreitol, 2% Servalyte pI 2-4 (Serva). For the resolution of protein samples a 10×12 cm gel electrophoresis system was used. For the identification of proteins 50-70 μg of proteins were applied to the sample template of a MALDI mass spectrometer (Voyager DEPRO, Perseptive). Peptide mass fingerprints were searched with the program MS-FIT (prospector.ucsf.edu/ucsfhtml/msfit.htm) using all cellular proteins in the NCBI data base allowing a mass accuracy of 100 ppm for the peptide masses. Partial enzymatic cleavages leaving two cleavage sites, oxidation of methionine, pyroglutamic acid formation at the N-terminal glutamine, and modification of cysteine by acrylamide were considered in these searches.

Apoptosis and Western blot analysis of CM incubated with patient sera or antibody was preformed as previously described

### REFERENCES

Applicants make no statement, inferred or direct, regarding the status of the following references as prior art. Applicants reserve the right to challenge the veracity of any statements made in these references, which are incorporated herein by reference.
Abdel-Aziz, G, et al. 1990. Am J Pathol. 137: 1333-42.
Alcolado, R, M. J. Arthur and J. P. Iredale. 1997. Clin Sci (Lond). 92: 103-12.
Ashkenazi, A. 2002. Nat Rev Cancer. 2: 420-30.
Barco, A, E. Feduchi and L. Carrasco. 2000. Virology. 266: 352-60.
Basu et al., Virology 298:53-62 (2002).
Blanco, R, L. Carrasco and I. Ventoso. 2003. J Biol. Chem. 278: 1086-93.
Choo, Q. L., et al. 1989. Science. 244: 359-362.
Clarke, B. 1997. J. Gen. Virol. 78: 97-2410.
DiBisceglie, A. M., R. L. Cairithers, and G. J. Gores. 1998. Hepatology. 28:1161-1165.
Du, C., et al. 2000. Cell. 102: 33-42.
Dufour, J. F., R. DeLellis, and M. M. Kaplan. 1997. Ann Intern Med. 127: 981-5.
Dufour, J. F, R. DeLellis, and M. M. Kaplan. 1998. Dig Dis Sci. 43: 2573-6.
Dziegielewska et al., J. BIOL. CHEM. 265:4354-4357 (1990).
Farci, P, et al. 1992. J Infect Dis. 165:1006-11.
Farci, P, et al. 1994. Proc Natl Acad Sci USA. 91:7792-6.
Fischer, R, et al. 2001. Gastroenterology. 120: 1212-26.
Fischer, R, et al. 2002. Gastroenterology. 123: 845-61.
Friedman, S. L, et al. 1985. Proc Natl Acad Sci USA. 82: 8681-5.
Friedman, S. L. 1993. N Engl J. Med. 328: 1828-35.
Friedman, S. L. 2000. J Biol. Chem. 275: 2247-50.
Ikeda, M, et al. 2003. Immunology. 108: 116-22.
Iordanov, M. S., et al. 2000. Cancer Res. 60: 1983-94.
Iredale, J. P, et al. 1998. J Clin Invest. 102: 538-49.
Iredale, J. P. 2001. Semin Liver Dis. 21: 427-36.
Issa R, et al. 2001. Gut. 48: 548-57.
Kothakota, S, et al. 1997. Science; 278: 294-298.
Li, M. L, et al. 2002. Virology. 293: 386-95.
Maher, J J, and R. F. McGuire. 1990. J Clin Invest. 86: 1641-8.
Marchenko, N. D, A. Zaika and U. M. Moll. 2000. J Biol. Chem. 275: 16202-12.
McGee, J. O, anD R. S. Patrick. 1972. Lab Invest. 26: 42940.
McGee, J, O, and R. S. Patrick 1972. J Pathol. 106:Pvi.
McHutchison, J. G, et al. 1998. N Engl J. Med. 339, 1485-92.
Prikhod'ko, G. G, et al. 2002. J. Virol. 76: 5701-10.
Purcell, R. H. 1994. FEMS Microbiol Rev. 14: 181-91.
Purcell, R. H. 1994. Proc Natl Acad Sci USA. 91: 2401-6
Ray, R. B., K. Meyer, and R. Ray. 2000. Virology. 271:197-204.
Ray, R. B. and R. Ray. 2001. FEMS Mini Review. 202: 149-156.
Rehermann, B., and F. V. Chisari. 2000. Curr Top Microbiol Immunol. 242: 299-325.
Rockey D. C., et al. 1992. J Submicrosc Cytol Pathol. 24: 193-203.
Roos, R. W, and T. Medwick. 1980. J Chromatogr Sci. 18: 626-30.
Saile, B, et al. 1997. Am J Pathol. 151: 1265-72.
Saito, I., et al. 1990. Proc. Nad. Acad. Sci., USA. 87: 6547-6549.
Shafee, N, and S. AbuBakar. 2003. J Gen Virol. 84: 2191-5.
Srinivasula, S. M, et al. 2000. J Biol. Chem. 275: 36152-7.
Srinivasula, S. M, et al. 2001. Nature. 410: 112-6.
Taimr, P, H. et al. 2003. Hepatology. 37: 87-95.
Verhagen, A. M, E. J. Coulson and D. L. Vaux. 2001. Genome Biol. 2, EVIEWS3009.
Verhagen, A. M, and D. L. Vaux. 2002. Apoptosis.7: 163-6.
Verhagen, A. M, et al. 2002. J Biol. Chem. 277: 445-54.
Wang, S, and W. S. E1-Deiry. 2003. Oncogene. 22: 8628-33.
Weiner, A. J., et al. 1992. Proc. Natl. Acad. Sci., USA, 89: 3468-3472.
Yu, C. L, and M. H. Tsai. 2001. Cancer Lett. 166: 173-84.
Zhang, X. D, et al. 1999. Cancer Res. 59, 2747-53.

What is claimed is:

1. A stellate cell death factor comprising a pro-apoptotic activity wherein the stellate cell death factor a) comprises a polypeptide having an amino acid sequence set forth in SEQ ID NO: 1, or fragments thereof, and b) is capable of inducing apoptosis in a liver stellate cell.

2. The stellate cell death factor of claim 1 wherein the stellate death factor is a polypeptide of approximately 23 kDa comprising amino acid residues 374-390 and 585-597 set forth in SEQ ID NO: 1.

3. The stellate cell death factor of claim 1 wherein the stellate death factor is a polypeptide of approximately 25 kDa comprising amino acid residues 342-346 and 374-390 set forth in SEQ ID NO: 1.

4. The stellate cell death factor of claim 1 wherein the stellate death factor is a polypeptide of approximately 46 kDa comprising amino acid residues 62-73, 342-346 and 374-390 set forth in SEQ ID NO: 1.

5. The stellate cell death factor of claim 1 wherein the stellate death factor is a polypeptide of approximately 50 kDa comprising amino acid residues 61-72 and 374-390 set forth in SEQ ID NO: 1.

6. The stellate cell death factor of claim 1 wherein the stellate death factor is a polypeptide having 93 percent homology with SEQ ID NO: 1 or fragments thereof, and capable of inducing apoptosis in a liver stellate cell.

7. The stellate cell death factor of claim 1 wherein the stellate death factor is a polypeptide comprising of at lease 30 continuous amino acids of the polypeptide set forth in SEQ ID NO: 1 and capable of inducing apoptosis in a liver stellate cell.

8. A fusion protein comprising the polypeptide fragment of claim 7 coupled to an immunogenic peptide.

9. A method of inhibiting the proliferation of a liver stellate cell, comprising contacting the liver stellate cell with an effective amount of a composition comprising a stellate cell death factor that is capable of inducing apoptosis in a liver stellate cell, wherein (a) the composition is comprised of an amino acid sequence set forth in SEQ ID NO: 1 or fragments thereof, and (b) the liver stellate cell dies.

10. The method of claim 9 wherein the composition is comprised of a polypeptide comprised of at lease 30 continuous amino acids set forth in SEQ ID NO: 1, and pro-apoptotic activity.

11. A method of claim 9 wherein the composition is a) incubated with immunoglobulin directed against an epitope on the stellate cell death factor, and b) pro-apoptotic activity is increased.

12. A method of claim 9 wherein the composition comprises a polypeptide of amino acid sequence set forth in SEQ ID NO: 1 or a fragment thereof, modified so as to elicit an auto-immune response from the host.

13. A method of claim 9 wherein the composition comprises a) a polypeptide of amino acid sequence set forth in SEQ ID NO: 1 or a fragment thereof, and b) an adjutant so as to elicit an auto-immune response from the host.

14. The method of claim 9 wherein the stellate cell is ex vivo.

15. The method of claim 9 wherein the stellate cell is a human stellate cell.

16. The method of claim 9 wherein the stellate cell is a LX2 cell.

17. (canceled)

18. A method of manufacturing a stellate cell death factor comprising the steps of applying the conditioned media to an anion exchange column, collecting a flow-through from the anion exchange column, applying the flow-through to a first cation exchange column, eluting a first fraction from the first cation exchange column with a buffer having approximately 0.5M NaCl, applying the first fraction to a second cation exchange column, and eluting a second fraction containing the stellate cell death factor using an increasing gradient of NaCl.

19. A method of manufacturing a stellate cell death factor comprising the steps of concentrating the conditioned media twenty-fold to produce a concentrated conditioned media; diluting the concentrated media with four volumes of buffer H, which consists of 20 mM Hepes, pH 7.4, 15% glycerol, to produce a primary buffered media; loading the primary buffered media onto a 2 ml Q-Sepharose column pre-equilibrated with buffer H; collecting a flow through fraction from the Q-Sepharose column; applying the flow through fraction onto a 2 ml SP-column; eluting a first fraction containing the stellate cell death factor from the SP-column with 5 ml of buffer H containing 0.5 M NaCl; dialyzing the first fraction containing the stellate cell death factor in buffer H to produce a buffered first fraction; loading the buffered first fraction onto an UNO-S column; eluting a second fraction from the UNO-S column using a linear gradient of 0 to 0.5 M NaCl in 20 ml of buffer H at a flow rate of 1 ml per minute, wherein the second fraction contains the stellate cell death factor.

20. A method of manufacturing the stellate cell death factor comprising of a) purifying the peptide forth in SEQ ID NO: 1, or fragments thereof and b) subjecting the peptide to enzymatic proteolysis such as to produce one or more smaller peptides capable of inducing apoptosis in a liver stellate cell.

21. (canceled)

22. A method of determining cirrhosis in a patient by measuring the patient's serum antibodies levels directed against fragments of the peptide forth in SEQ ID NO: 1.
